(19) United States
(12) Patent Application Publication
(10) Pub. No.: US 2024/0123108 A1
(43) Pub. Date: Apr. 18, 2024

(54) AIR SANITIZATION

(71) Applicant: Singletto Inc., Seattle, WA (US)

(72) Inventor: James Chongchu Chen, Clyde Hill, WA (US)

(21) Appl. No.: 18/379,863

(22) Filed: Oct. 13, 2023

Related U.S. Application Data
(60) Provisional application No. 63/415,902, filed on Oct. 13, 2022.

(51) Int. Cl. A61L 9/20

(52) U.S. Cl. CPC A61L 9/205; A61L 2209/15

(57) ABSTRACT

An apparatus and method to actively purify and sanitize air. A flow of air is directed over a surface that includes a photosensitizer formulation which is activated by light to emit singlet oxygen that inactivates and/or kills pathogens in the air flow. A fan device is employed to generate and direct the flow of air. The fan device includes the photosensitizer formulation disposed on fan blades, rotatable fan shafts, and interior surfaces of a housing. The flow of air is emitted towards an exterior surface. Activation of the photosensitizer by light emits singlet oxygen in the flow of air to inactivate pathogens disposed on the interior surface of the housing and the exterior surface.

### CROSS-REFERENCES TO RELATED APPLICATIONS

This application is a Utility patent application based on previously filed U.S. Provisional Patent Application No. 63/415,902, filed on Oct. 13, 2022, the benefit of the filing date of which is hereby claimed under 35 U.S.C. § 119(e) and the contents of which is herein incorporated by reference in its entirety.

### BACKGROUND

Air purification devices and equipment are important for health maintenance especially when pathogens contaminate the air such as has, and is occurring as a result of the SARS-CoV2-2 outbreak. Other respiratory pathogens such as influenza, respiratory syncytial virus, measles, monkeypox, rhinoviruses, mumps, hantavirus, and other pathogens can be transmitted through the air especially during outbreaks, along with chemical and biological agents deliberately or accidentally released as part of bioterror events or laboratory accidents.

Mitigations measures such as ultraviolet light or ozone emitting devices have downsides including tissue toxicity risks. Air filters that are of high filtration capability such as HEPA filters can require powerful noisy fans to push air though dense materials, and as such can be large, bulky devices if used for large volume spaces such as rooms. Filters and other devices such as electrostatic precipitators may only trap airborne pathogens, and do not inactivate or actively kill the pathogens. Clearly, there is a need for improved air purification that actively inactivates and/or kill pathogens in the air, without the toxicity risks to the eyes and skin that can be caused by ultraviolet light, or lung, eye and other toxicity risks caused by ozone production.

### SUMMARY

The features for one or more embodiments may include one or more of the following limitations:
A tangential fan design that creates a curtain of disinfected air that can be directed anterior to the face of a user of the device;
Fan blades comprised of a material such as synthetic or natural fibers that easily and readily accept at least one photosensitizer and other substances;
A miniature rechargeable electric motor which enable rotation of the shaft that the fan blades are attached to;
A shaft which is comprised of a polymer or a metal, which is stiff or flexible, enabling the tangential fan device to be configured into a curved shape if desired;
Fan blades that are optionally flexible, or that incorporate stiffening struts;
Variable speed electric motor that can rotate the fan blades slower or faster;
Fan blade configuration that is flat, curved, or in a screw shape;
External housing for the fan shaft/blade assembly that incorporates at least one photosensitizer;
A light source which may be ambient light, or an incorporated light source such as at least one light emitting diode, or which may also incorporate non-imaging lenses to direct light onto the fan blades and the interior of the housing incorporating at least one photosensitizer;
Fan blade number can range from two opposing blades to 20 blades spaced apart uniformly;
Fan blade rotation speed can vary from 10 to 100,000 revolutions per minute;
Fan blade dimensions can range from 1.0 cm to 500.0 cm in length and 1.0 cm to 10.0 cm in width;
Fan blade and housing optionally incorporate phase change materials for heating and cooling of expelled air;
A reservoir which is refillable or exchangeable contains at least one photosensitizer formulation, is used to recharge the fan blade(s) and inner surface of the fan housing;
The cylindrical housing coated with at least one photosensitizer can also rotate around an axis;
An ultrasonic motor can be utilized to spin the fan blades; and/or
Techniques of incorporating at least one photosensitizer into a fabric or surface.

### VARIOUS EMBODIMENTS

In one or more embodiments, the tangential fan housing is lined with natural and/or synthetic fibers capable of accepting at least one photosensitizer formulation. Further, the one or more embodiments may incorporate at least one photosensitizer which non-exclusively may include known and contemplated photosensitizers including but not limited to all types of methylene blue derivatives and methylene blue itself, chlorophyll derivatives, tetrapyrrole structures, porphyrins, chlorins, bacteriochlorins, phthalocyanines, texaphyrins, prodrugs such as aminolevulinic acids, phenothiaziniums, squaraine, boron compounds, various transition metal complexes, hypericin, riboflavin, curcumin, titanium dioxide, psoralens, tetracyclines, indocyanine green, flavins such as riboflavin, erythrosine, and the like. The one or more embodiments may also include known or contemplated nano-compositions of photosensitizers and photosensitizers linked to a variety of other substances, or utilized in combination with other substances which may improve the photodynamic decontaminating effect. Also, in the one or more embodiments, the fan blade material is coated with known scented oils with known antimicrobial properties such as tea tree oil, lemongrass oil, eucalyptus oil, menthols, peppermint oils, lemongrass oils, orange oils, various aldehydes, phenols, and the like, which may be applied as a spray. Light sources enabling photoactivation include can include, but are not limited to lasers, laser diodes, light emitting diodes, and other semiconductor light sources such as those using quantum dot technology. Fluorescent, incandescent lighting, ambient outdoor light, light from combustion of materials causing flame, are also included in the present disclosure. Moreover, in the one or more embodiments, the light source is comprised of at least one light emitting diode which emits a light spectrum which substantially overlaps the absorption band of at least one photosensitizer, enabling rapid and efficient photoactivation.

Additionally, in the one or more embodiments, the rotating shaft with attached fan blades is comprised of a flexible optical fiber with an outer diameter of 0.1 millimeters to 8.0 millimeters, with a bend radius of 1.0 centimeters. In another embodiment the optical fiber is butted up to at least one light emitting diode, and is able to leak light along its entire length, providing photoactivating light to the fan blades coated with at least one photosensitizer. In yet another of the one or more embodiments, the rotating shaft is 3.0 centimeters to 50.0 centimeters long, and may optionally be in a curved or even a coiled shape, with a bend radius of 5-10 centimeters, or greater.

Further, in the one or more embodiments, the tangential fan housing may be in the shape of a cylinder, with an outer diameter of 1.0 centimeter to 50.0 centimeters or greater, and may encompass a length of 3.0 centimeters to 100.0 centimeters or longer. In yet another of the one or more embodiments, the tangential fan housing is 0.1 millimeter to 10.0 millimeters in thickness.

Also, in one or more of the embodiments, the cylindrical housing coated with at least one photosensitizer can also rotate around an axis.

Further, in the one or more embodiments, the interior of the tangential fan housing is coated with a reflective coating comprised of silver, gold, or aluminum, or a film such as mylar, which reflects photoactivating light back to the fan blade surfaces coated with at least one or more photosensitizers.

In one or more embodiments, the concentration range of at least one photosensitizer can range from 0.1 micromolar to 2000 micromolar, or up to a 5% solution or up to a 75% weight for weight aqueous formulation.

Also, in the one or more embodiments, at least one light source is incorporated into the tangential fan housing. Further, in yet another of the one or more embodiments, at least one non-imaging lens is incorporated into the tangential fan housing that admits photoactivating ambient light into the interior of the housing.

Further, in the one or more embodiments, a substance which can attract water vapor from the atmosphere such as hydroscopic konjac gum may be incorporated into the fan blade surface and interior of the fan assembly housing along with at least one photosensitizer to provide an air cooling adiabatic mechanism, along with pathogen inactivation and killing, with air cooling enabled by air flow over the hydroscopic substance which induces evaporation of the water in the hydroscopic substance.

In the one or more embodiments, a refillable or replaceable reservoir containing at least one photosensitizer formulation is incorporated into the tangential fan housing, which elutes the formulation in a controllable manner onto the fan blades and into the lining of the tangential fan housing. This action renews the photosensitizer that may photobleach over time, which can reduce effective photoactivation from light absorbed by the photosensitizer, and which in turn may reduce the antimicrobial or antitoxin and/or anti-poison action of the photosensitizer.

In the one or more embodiments, a tangential fan housing may incorporate a hinged or removable segment, which permits reapplication of at least one photosensitizer formulation, applied as a film, a powder, or a spray, directly into the interior of the housing, coating the fan blade(s) and the interior of the housing. This action can renew capability of the tangential fan to induce the photoactive disinfection/sanitization/inactivation processes.

In one or more embodiments, the tangential fan housing may be comprised of an optically clear polymer such as, but not limited to, transparent polypropylene, polystyrenes, polycarbonate, polyethylene, or polyesters which can transmit photoactivating light to the interior of the tangential fan housing. Further, in the one or more embodiments, a light source can be positioned external to a light transmissible housing. Also, in another of the one or more embodiments, a light source may be positioned internally within the light transmissible housing.

In one or more embodiments, an ultrasonic motor, which is a type of piezoelectric motor powered by vibration of a stator abutting a rotor, can be utilized to spin the fan blades.

Also, in one or more embodiments, an active powered fan may be used to passively spin a secondary fan proximate and aligned parallel to the main powered fan, using air flow from the spinning powered fan to turn the secondary fan blade. The fan blade(s) surfaces and the inner housing surfaces of the dual housing arrangement may incorporate at least one photosensitizer, enabling two points of photosensitizer contact in sequence to improve the air disinfection capability. Further, the passively driven secondary fan can minimize impedance of air flow from the main powered fan when compared to a conventional filter.

Also, in one or more embodiments, at least two tangential fans are juxtaposed in parallel, with one fan rotating in one direction and another proximate fan rotating in an opposite direction, to increase air contact time with each fan blade and the fan blade housing, with concomitant increased light exposure. In yet one or more of the embodiments, one of the two juxtaposed fans may be larger in diameter, at least 1.5 to 2× or greater in diameter, such that air is ejected at a particular direction tangential to the long axis of the tangential fan assembly, while still providing for a net increase in air contact time with the fan blades and fan blade housing. The counter-rotation scheme essentially permits air recirculation and increases exposure of potentially contaminated air to reactive oxygen species and singlet oxygen by the illuminated photosensitizer(s) coating the fan blades and the inner housing of the fan assembly.

Further, in one or more embodiments, the fan blades of a rotating fan may be of a width of 0.1 millimeter to 2.0 centimeters, spaced apart on a rotating shaft which is comprised of a flexible polymer, housed in a flexible optionally transparent housing which is tube-like in structure, the entire structure able to be bent into a curvilinear shape as desired by a user.

Also, in one or more embodiments, a tangential fan assembly may be configured in a curved shape that partially encircles a hat with a brim, at the junction of the brim with the inferior aspect of the dome of the hat, which contains a slit which permits the flow of air in a direction inferior to the hat brim, creating an air curtain anterior to the face. Further, a zone of disinfected or detoxified or otherwise purified air may be created anterior to the face, enabling inspiration of pathogen, allergen, and toxin free air, while protecting the eye surfaces, nose, mouth, mucous membranes contacting the air stream, and the facial skin from contamination.

In one or more embodiments, a photoactive fabric incorporated into the fan blade(s) and/or fan blade(s) surface is rough, or textured, or contains micro-projections/micro-loops such as may be found in microfiber type of materials in order to maximize the surface area of the fan blade(s) surface which allows for maximal loading of at least one photosensitizer on the fan blade(s) surface which in turn generates increased amounts of reactive oxygen species when exposed to light of the proper waveband or wavelength absorbed by the photosensitizer(s).

In one or more embodiments, a fabric or coating may incorporate at least one or more photosensitizers such as methylene blue at a concentration ranging from 1.0 micromolar to 3,000 micromolar. The photosensitizer may be incorporated using a variety of methods such as heated bath dyeing, sublimation, jet pressure and drying application, heated transfer sheet processing, roller application with heat and pressure, or the like. Further, in one or more embodiments, a photo-linking process for surface attachment of photosensitizers can be used, as well as modifications to the above processes using chemicals such as sodium hydroxide that increase the pH of the solutions, which can increase the photosensitizer uptake into a fabric or coating which is incorporated into the fan blades, and/or fan housing interior.

Unexpectedly, use of one or more of the above embodiments for techniques to apply water soluble photosensitizer(s) such as methylene blue and other hydrophilic or amphiphilic photosensitizers to hydrophobic materials such as polypropylene, or to hydrophobic coatings such as silicones, siloxanes, waxes, acrylics, methylmethacrylates, urethanes, fluorinated compounds, and the like, can lead to aggregation of photosensitizers which is known to reduce singlet oxygen production and reduce antimicrobial efficiency.

Unexpectedly, in one or more embodiments, the known reduction of antimicrobial action by stacking of planar photosensitizers such as methylene blue, leading to dimer, trimer, and greater numbers of “piled” configurations does not occur on the dry, hydrophobic or non-hydrophobic materials and surfaces. Spraying, dipping, wiping, and similar application methods of very high photosensitizer (such as methylene blue and other known or contemplated light activated substances) concentrations in solution at or above 1000 micromolar to hydrophobic surfaces and materials such as polypropylene followed by room temperature or less than 100 degrees F. drying for at least two hours in ambient pressure environments (one atmosphere) unexpectedly leads to successful incorporation and embedding of high concentrations of water soluble or amphiphilic photosensitizers into hydrophobic materials, which teaches away from the prior art/notion that these types of materials such as polypropylene are virtually impossible to dye without special dispersal agents, dye modifications, hydrophobic surface modifications, and extreme temperature and pressures.

The above processes of the one or more embodiments, represent a form of photo-crosslinking of at least one photosensitizer to the hydrophobic coating or material or substance using ambient indoor light, typically less than 50,000 lux, and more often less than 100 lux.

Furthermore, in one or more embodiments, an unexpected benefit of the above photosensitizer incorporation methods and techniques may be reduction of photobleaching phenomenon which occurs from auto-destruction by singlet oxygen, or photodegradation. Photobleaching reduces the antimicrobial action from light absorption by intact photosensitizer molecules, since there are fewer photoactive photosensitizer molecules. The stacking of photosensitizer molecules or monomers creating dimers, trimers, and greater numbers of “piled” molecules (multimers) unexpectedly leads to photobleaching resistance probably due to reduced light adsorption by the less exposed photosensitizer molecules which are in essence protected by light. Despite less net photoactivation processes occurring in the relatively dry state, at comparable concentrations, compared to an aqueous solution, the antimicrobial action remains comparable, which teaches away from the known art. In one or more embodiments, photomicrographic image(s) captured after methylene blue application demonstrate methylene blue multimer aggregates which are unexpectedly highly resistant to photobleaching and yet capable of generating effective antimicrobial singlet oxygen amounts, which teaches away from prior art that dimers, trimers, and multimers are less effective than photosensitizers in solution. In the one or more embodiments, tests comparing the equivalent methylene blue concentrations in monomeric solutions and on hydrophobic materials as multimers such as polypropylene as the hydrophobic material can be performed demonstrating that multimeric photosensitizer multimer formations are effective as antimicrobials.

In the one or more embodiments, pre-treatment of materials may be provided with at least one photosensitizer in solution or in a dry powder state, including techniques such as jet spraying, dipping and nipping, high pressure roller and temperature baths, sublimation, heat transfer sheet, increased pH, photo-crosslinking, and other known dye application processes, followed by spray and/or dipping, and/or wiping applications of at least one photosensitizer in solution, or applied as a dry powder can unexpectedly augment the incorporated photosensitizer concentration and volume on a material or surface, which may be hydrophobic, and not normally able to accept a high photosensitizer or dye concentration, above 1000 micromolar, or even above 50 micromolar.

In the one or more embodiments, a flexible or conformable tangential fan may be affixed or incorporated into a pair of eye glasses, goggles, a headband, or into a helmet (positioned anterior to the face and head), or related headgear and/or eyewear. In the one or more embodiments, the flexible or conformable tangential fan can be configured as a necklace shape and worn around the neck, with the airstream directed in a superior or upward direction toward the face of the user. In these relatively thin embodiments, the fan blade housing diameter may be 5.0 mm or less, and the fan shaft can be optionally flexible and conformable into a curved shape, and the fan shaft driving mechanism and battery may be incorporated into a frame of eye glasses, goggles, headband, helmet, and/or a necklace. In these one or more embodiments, a rotating shaft with attached fan blades may be driven by a miniature electric motor which is powered by a rechargeable battery.

In one or more embodiments, one or more fan blades may be replaceable, or may be anchored in place on a rotating polymeric or metallic shaft using epoxies, cyanoacrylates, polyurethanes, or other known bonding agents and glues. In the one or more embodiments, the flexible rotating shaft may be comprised of nitinol. Further, in the one or more embodiments, the fan blade rotation speed can be variable and adjustable, which can optimize contaminated air contact time for decontamination/treatment and expelling parameters for maximal inhalation of decontaminated/treated air. Further, in the one or more embodiments, the rotating fan speed may be alternately increased and decreased, to provide for more photo-disinfection/photoinactivation time at slower rotation speeds, followed by more rapid rotation speeds which ejects the disinfected/purified air at a faster rate, increasing the probability of a user inhaling the photo-processed air.

In one or more embodiments, a series of experiments may be performed using aerosolized microbes, smoke, and other noxious airborne substances and organisms to determine optimal fan speeds, photosensitizer(s) concentrations, air dwell time within a fan assembly housing, and the like, by performing appropriate assays demonstrating decontamination and/or breakdown or destruction of noxious airborne substances, particles, or pathogens or surrogate microbes. Further, in the one or more embodiments, a wetting agent such as water applied or captured from the air to the fan blades to facilitate capture and decontamination and/or breakdown of noxious airborne substances or microbes which can be dissolved or attracted to the fan blade surfaces for photoinactivation by light and at least one photosensitizer disposed on the fan blade(s) surface or inner surface of the fan assembly housing, or within an incorporated nozzle or air flow director or tube like air flow director.

In one or more embodiments, at least one of known phase-change materials including polymers, salts, and other known chemicals, known to change from solid-liquid and vice versa, may be optionally part of this disclosure. These materials may be microencapsulated, and can be incorporated into the fan blades and/or the tangential fan housing, to provide heated or cooled expelled purified air as desired, for hot and cold environments. Further, in the one or more embodiments, known phase change materials may be incorporated into the substance of the fan blade(s). Also, in the one or more embodiments, phase change materials may non-exclusively include waxes such as eicosane, octadecane, nonadecane, heptadecane, and hexadecane, which are known to be microencapsulated for incorporation into fan blade(s). Other phase change substances include paraffin wax, carboxylic acids, Glauber's salt which is sodium sulfate decahydrate, and calcium chloride hexahydrate. Matrix coating using coating substances such as polyurethanes, acrylics, and the like, various knifing techniques, padding techniques, dip and transfer coating, gravure processing, and foam dispersion using polyurethanes, as examples of application and incorporation methods and techniques known in the textile and fabric manufacturing industry, can be used to coat fan blade(s).

### DRAWING DESCRIPTIONS

FIG. 1A depicts elongate shaped fan blade A, with a width A1 that is greater than its height A2, which is comprised of a polymer, which may be a synthetic material, and/or a natural fiber.

FIG. 1B shows a side view of fan blade A3 as curved in its height A2 dimension.

FIG. 1C illustrates fan blade A4 in the shape of a screw or auger, longer in width A1 than height A2.

FIG. 1D shows a polymeric or metallic elongate shaft B, to which fan blade A is affixed with epoxy, resin, or glue.

FIG. 1E depicts replaceable blade A4 shown in a position for placement into slot B1 shown in a side view in elongate shaft B2 also shown in side view.

FIG. 1F shows tangential fan housing C in an elongate configuration, lined with coating C1, which may be comprised of at least one photosensitizer and/or phase change material/and or reflective material.

FIG. 1G depicts tangential fan housing alternative C2 is comprised of a transparent material, admitting light D from external light source D1. Incorporated light source D2 which is optionally at least one light emitting diode, can also supply light D to interior of tangential fan housing C2.

FIG. 1H shows rechargeable battery D3 powers electric motor D4 incorporated into tangential fan housing C which drives rotating shaft D5 which rotates attached fan blade A3.

FIG. 1I depicts A3a as a representation of a segmented multiple fan assembly attached to rotating shaft D5.

FIG. 1J shows a side view of curved fan blade A3 coated with at least one photosensitizer and/or phase change material E.

FIG. 1K depicts a side view of a curved shape of rotating shaft D5a that is attached to curved fan blade A3a.

FIG. 2A shows microencapsulated phase change material E1 surrounded by microcapsule E, which ranges in outer diameter from 1.0-500.0 microns.

FIG. 2B depicts spray bottle F2 delivering photosensitizer aerosol F3 into the interior of housing F through open hinged cover F1 into opening F4.

FIG. 3A shows dashed lines G which represent a rotatable fan shaft powered by battery/driver mechanism G1 incorporated into glasses temple G2. Furthermore, rotating shaft G with attached fan blade(s) G6 which incorporates at least one photosensitizer, is contained in housing G3 which constitutes the inferior aspect of the eye glasses frame G4 which includes temple G2. The opening in housing G3 where disinfected air is ejected is located at the inferior aspect of housing G3, enabling the disinfected air stream to be directed toward mouth and nose G5.

FIG. 3B illustrates fan shaft G is comprised of an optical fiber G7, which is butted up to light emitting diode array G8 which emits light that is powered by rechargeable battery G9. Light which leaks from optical fiber G7 along its length serves to photoactivate at least one photosensitizer incorporated into fan blade(s) G6, attached along the length of optical fiber G7.

FIG. 3C shows a cross section of fan shaft G with attached fan blades G6 proximate and in parallel to secondary fan shaft G10, which is rotated by air flow G11 from powered fan shaft G turning fan blades G6 pushing against secondary fan blades G12.

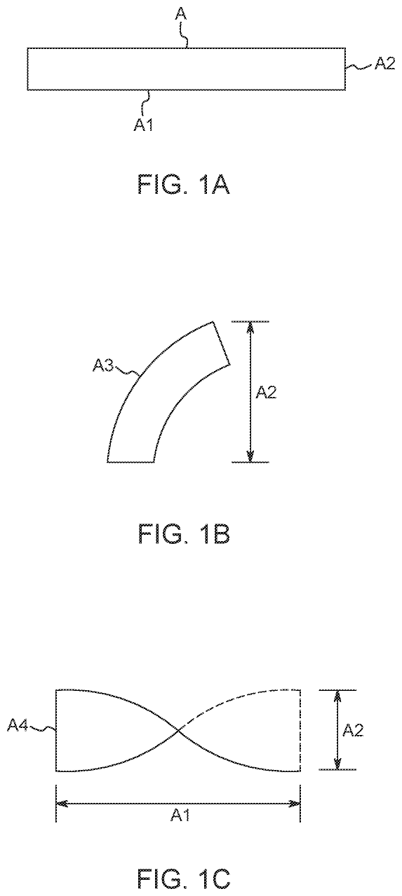

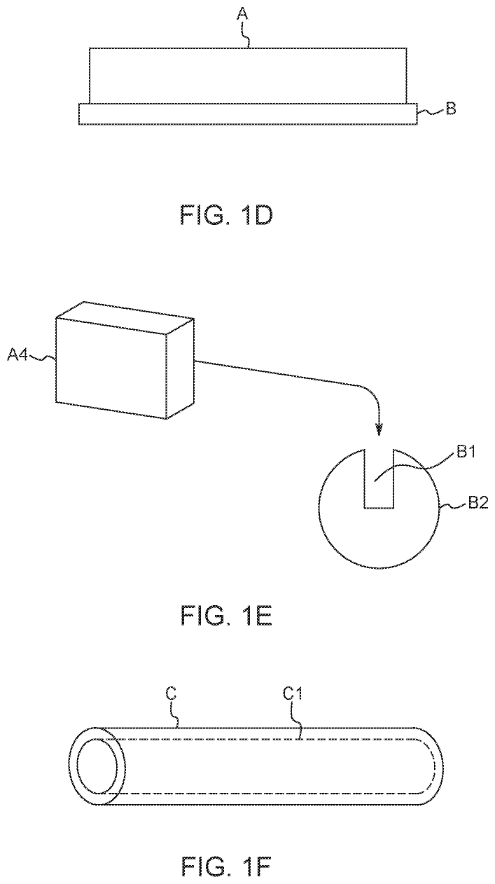

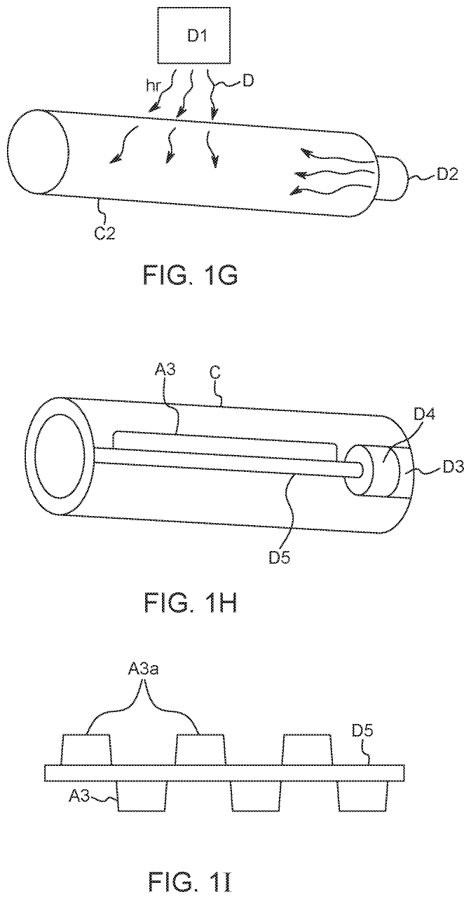

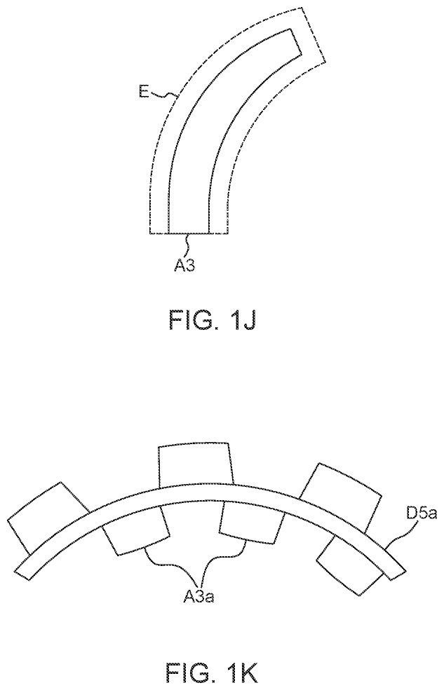

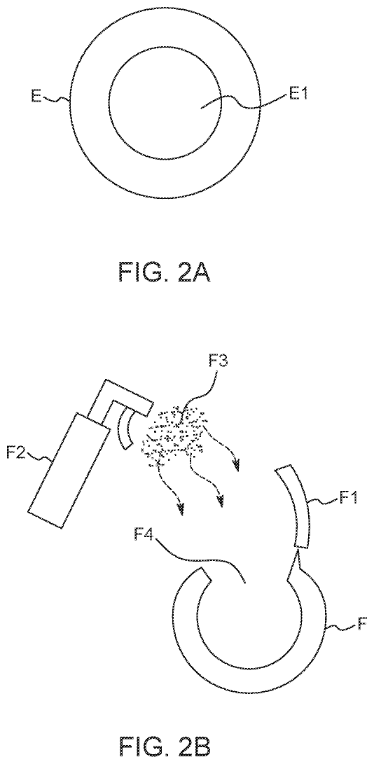

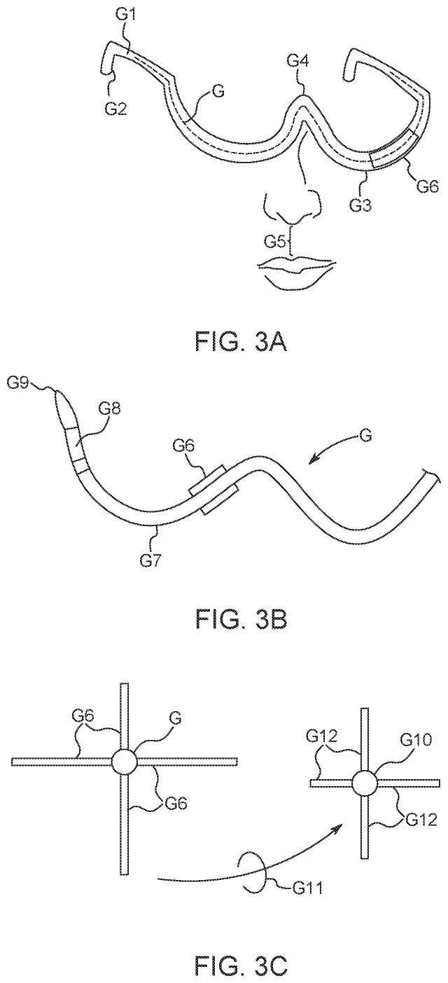

What is claimed as new and desired to be protected by Letters Patent of the United States is:

1. A method, comprising:
employing a fan device to generate a flow of air, wherein the fan device includes a photosensitizer formulation that is disposed on one or more of a fan blade, a rotatable fan shaft, and an interior surface of a fan housing;
directing the flow of air towards the one or more of the fan blade, rotatable fan shaft, the interior surface of the fan housing and an exterior surface; and
employing light to activate the photosensitizer formulation disposed within the fan device, wherein the activated photosensitizer emits singlet oxygen in the flow of air to inactivate pathogens disposed on the one or more of the fan blade, rotatable fan shaft, the interior surface of the fan housing and the exterior surface.

2. The method of claim 1, further comprising:
employing one or more secondary fan blades and a rotatable secondary fan shaft to relay the directed flow of air towards the external surface.

3. The method of claim 2, further comprising:
disposing the photosensitizer formulation on the one or more secondary fan blades and the secondary rotatable fan shaft; and
employing the light to activate the photosensitizer formulation disposed on the one or more secondary fan blades and the rotatable secondary fan shaft to emit singlet oxygen in the flow of air.

4. The method of claim 1, wherein the one or more fan blades of the fan device form a screw shape.

5. The method of claim 1, wherein the rotatable fan shaft is configured to include one or more curved shapes.

6. The method of claim 1, wherein employing the light to activate the photosensitizer further comprises:
activating one or more portions of the photosensitizer formulation that is disposed on the external surface.

7. The method of claim 1, wherein the one or more fan blades, further comprise:
one or more shapes for a fan blade that is disposed contiguously along a length of the rotatable fan shaft.

8. The method of claim 1, wherein the one or more fan blades, further comprise:
one of more shapes for a fan blade that is disposed intermittently along a length of the rotatable fan shaft.

9. The method of claim 1, further comprising:
employing a frame for one of a pair of eye glasses, goggles, a headband, a helmet or a necklace to enclose the fan device; and
wherein the activated photosensitizer emits singlet oxygen in the flow of air that is emitted from the frame towards a face of a user.

10. The method of claim 1, further comprising:
employing one or more ports in the fan housing to reapply the photosensitizer formulation to the one or more of a fan blade, the rotatable fan shaft, and the interior surface of the fan housing.

11. A fan device, comprising:
one or more fan blades;
a rotatable fan shaft;
a fan housing;
a photosensitizer formulation that is disposed on one or more of the fan blade, the rotatable fan shaft, and an interior surface of the fan housing; and
wherein the fan device generates a flow of air that is directed towards an exterior surface; and wherein the photosensitizer formulation is activated by light to emit singlet oxygen in the flow of air to inactivate pathogens disposed on the one or more of the fan blade, the rotatable fan shaft, the interior surface of the fan housing and the exterior surface.

12. The fan device of claim 11, further comprising:
one or more secondary fan blades and a rotatable secondary fan shaft that are operable to relay the directed flow of air towards the external surface.

13. The fan device of claim 12, wherein the photosensitizer formulation is disposed on the one or more secondary fan blades and the secondary rotatable fan shaft; and wherein the light activates the photosensitizer formulation disposed on the one or more secondary fan blades and the rotatable secondary fan shaft to emit singlet oxygen in the flow of air.

14. The fan device of claim 11, wherein the one or more fan blades of the fan device form a screw shape.

15. The fan device of claim 11, wherein the rotatable fan shaft is configured to include one or more curved shapes.

16. The fan device of claim 11, wherein activating the photosensitizer further comprises:
activating one or more portions of the photosensitizer formulation that is disposed on the external surface.

17. The fan device of claim 11, wherein the one or more fan blades, further comprise:
one or more shapes for a fan blade that is disposed contiguously along a length of the rotatable fan shaft.

18. The fan device of claim 11, wherein the one or more fan blades, further comprise:
one of more shapes for a fan blade that is disposed intermittently along a length of the rotatable fan shaft.

19. The fan device of claim 11, further comprising:
a frame for one of a pair of eye glasses, goggles, a headband, a helmet or a necklace to enclose the fan device; and
wherein the activated photosensitizer emits singlet oxygen in the flow of air that is emitted from the frame towards a face of a user.

20. The fan device of claim 11, further comprising:
one or more ports in the fan housing that are operable to reapply the photosensitizer formulation to the one or more of a fan blade, the rotatable fan shaft, and the interior surface of the fan housing.

21. The fan device of claim 11, further comprising:
one or more antimicrobial formulations that are disposed on the one or more of the fan blade, the rotatable shaft or the interior surface of the fan housing, wherein the one or more antimicrobial formulations include one or more of tea tree oil, lemongrass oil, eucalyptus oil, menthol oil, peppermint oil, lemongrass oil, orange oil, aldehyde, and phenol.

22. The fan device of claim 11, further comprising:
one or more light sources to emit the light for activation of the photosensitizer formulation, wherein the one or more light sources include a laser, a laser diode, a light emitting diode, a fluorescent lamp, an incandescent lamp, ambient outdoor light, and wherein one or more portions of the one or more light sources emit a light spectrum that provides an overlap of an absorption band of the photosensitizer formulation.
